(19) United States
(12) Patent Application Publication
(10) Pub. No.: US 2025/0235465 A1
(43) Pub. Date: Jul. 24, 2025

(54) USE OF SODIUM TRANS-[TETRACHLORIDOBIS(1H-INDAZOLE)RUTHENATE(III)] TO AMELIORATE PROTEASOME INHIBITOR RESISTANCE

(71) Applicant: Bold Therapeutics Inc., Vancouver (CA)

(72) Inventors: Paromita RAHA, Vancouver (CA); Mark BAZETT, Vancouver (CA); Edward Russell MCALLISTER, Vancouver (CA); James PANKOVICH, Vancouver (CA); Lisa CRAWFORD, Belfast, Antrim (GB)

(21) Appl. No.: 18/705,438

(22) PCT Filed: Oct. 24, 2022
(86) PCT No.: PCT/CA2022/051569

§ 371 Date: Apr. 26, 2024

Related U.S. Application Data
(60) Provisional application No. 63/272,979, filed on Oct. 28, 2021.

(51) Int. Cl. A61K 31/555; A61K 31/407; A61K 31/454; A61K 31/5377; A61K 31/69; A61K 38/07; A61P 35/00

(52) U.S. Cl. CPC A61K 31/555; A61K 31/407; A61K 31/454; A61K 31/5377; A61K 31/69; A61K 38/07; A61P 35/00

(57) ABSTRACT

Methods and corresponding uses are provided for treating disease in a patient, involving the use of sodium trans-[tetrachloridobis(1H-indazole)ruthenate(III)] so as to ameliorate drug resistance, including resistance to proteasome inhibitors and immunomodulatory imide drugs. The disease may for example be relapsing/refractory multiple myeloma.

### FIELD

The invention is in the field of therapeutics, including the combined use of sodium trans-[tetrachloridobis(1H-indazole)ruthenate(III)] and proteasome inhibitors, for example in treating cancers.

### BACKGROUND

Sodium trans-[tetrachloridobis(1H-indazole)ruthenate(III)] is a coordinated complex of ruthenium having anticancer activity (also known as BOLD-100, KP1339, NKP-1339, IT-139, and Na [RullICI4 (Hind) 2]). Methods of making alkali metal salts of trans-[tetrachlorobis(1H-indazole) ruthenate (III)] are for example described in PCT Patent Publication WO2018204930, such compounds having Formula I:

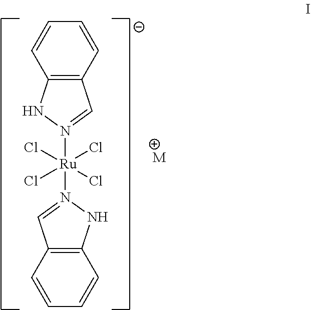

wherein M is an alkali metal cation, including the sodium salt:

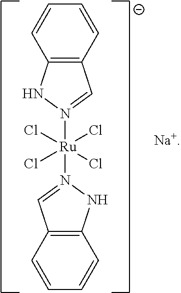

Myeloma is a lymphoid malignancy involving plasma cells primarily resident in the bone marrow, although malignant plasma cells may also be seen in peripheral blood, soft tissue and organs. Myeloma is accordingly a plasma cell dyscrasia, which manifests in forms that may be distinguished by the affected sites, in multiple myeloma several different areas are affected, in plasmacytoma only one site is affected, in localized myeloma adjacent sites are affected, and in extramedullary myeloma there is involvement of tissue other than bone marrow. As used herein, the term “myeloma” accordingly refers to the spectrum of diseases recognized in the art as such. Within this spectrum of disease, relapsed MM is generally regarded as a recurrence of the disease after prior response, typically based on objective clinical criteria, and relapsed/refractory MM (RRMM) is generally defined as a disease which becomes non-responsive or progressive on therapy or within 60 days of the last treatment in patients who had achieved a minimal response (MR) or better on prior therapy. The treatment of RRMM poses particular challenges.

Immunomodulatory imide drugs (IMiDs) have been used in the treatment of myeloma, including the IMiD drugs thalidomide and its analogues (which include lenalidomide, pomalidomide, and iberdomide). These drugs are understood to act as modulators of the protein Cereblon.

The proteasome is a protease complex that mediates selective hydrolysis of proteins, in concert with ubiquitin acting as a marker for regulated proteolysis, in both transformed and normal eukaryotic cells. The use of proteasome inhibitors (PIs) has been transformative in the treatment of multiple myeloma (MM), mantle-cell lymphoma (MCL) and amyloidosis. PIs are understood to have antiproliferative and antitumor activities mediated by inhibiting the proteasomal degradation of proteins by targeting a variety of proteasomal components, with distinctions recognized between inhibitors of the constitutive proteasome expressed in most cell types and the immunoproteasome expressed in immune cells. Clinical activity of PIs has also been reported in Waldenstrom macroglobulinemia, T-cell lymphoma amyloidosis, and other lymphoproliferative disorders, such as lymphoid malignancies. Three proteasome inhibitors bortezomib, carfilzomib and ixazomib citrate are in use for treating multiple myeloma and in trials for additional uses. Marizomib, oprozomib, and delanzomib are PIs that have been used in clinical trials for MM as well as other indications.

Immunoproteasome inhibitors include ONX-0914 and KZR-616, and are for example described in WO2006099261, WO2006092326, WO2006045066, WO2007149512, WO2010108172, WO2014152134, WO2014152127, WO2011109355, WO2014056954, WO2014056748. With the increasing success of PIs in treating a number of diseases, has come the growing problem of resistance to proteasome inhibitors.

### SUMMARY

Methods and corresponding uses are provided for ameliorating therapeutic resistance to a proteasome inhibitor (PI) in a patient in need thereof, comprising administering an effective amount of sodium trans-[tetrachloridobis(1H-indazole)ruthenate(III)](BOLD-100). Methods are similarly provided for treating a cancer or a lymphoproliferative disorder in patient in need thereof, comprising administering an effective amount of BOLD-100 in combination with a combinatorially effective amount of a proteasome inhibitor (PI). A combinatorially effective amount of a PI is a PI dosage or regimen in which the therapeutic benefit of the PI is improved by the combined treatment with BOLD-100. The disease or disorder amenable to combined treatment with BOLD-100 and a PI may for example be a disease that is susceptible to developing resistance to treatment with the PI in the absence of treatment with BOLD-100. The use of BOLD-100 so as to ameliorate therapeutic resistance to the proteasome inhibitor may accordingly be prophylactic, in the sense of ameliorating the transition to a PI resistant disease state.

The effective amount of BOLD-100 and the combinatorially effective amount of the PI may for example be additive or synergistically effective, where synergy is assessed by any one of a variety of art recognized metrics. The disease may be resistant to treatment with the PI alone. The disease may be a multiple myeloma (MM), such as a recurring refractory MM (RRMM). A select embodiment accordingly involves the treatment of RRMM, where the RRMM has acquired resistance to a PI, and wherein the treatment comprises administration of the PI in combination with BOLD-100, where the BOLD-100 is used in an amount effective to overcome or ameliorate the acquired resistance to the PI. BOLD-100 and the PI may be administered sequentially, in any order, or administered in combination, in a co-formulation or separately.

Methods are also provided for treating a myeloma that is resistant to a therapeutic, comprising administering to the patient an effective amount of sodium trans-[tetrachloridobis(1H-indazole)ruthenate(III)], wherein the therapeutic is effective to treat myelomas that are not resistant. The therapeutic may for example be a PI, an immunomodulatory imide drug, a cereblon modulator drug, thalidomide or a thalidomide analogue (such as lenalidomide, pomalidomide or iberdomide).

### BRIEF DESCRIPTION OF THE DRAWINGS

FIG. 1 is a line graph illustrating the anticancer activity of sodium trans-[tetrachloridobis(1H-indazole)ruthenate(III)](BOLD-100), showing inhibition of growth of various Multiple Myeloma (MM) cancer cell lines.

FIG. 2 includes two line graphs, illustrating: (2A) the growth curves of cell lines MM.1S and MM.1S BR in presence of increasing doses of BOLD-100 over 24 hours, demonstrating higher sensitivity of the PI-resistant line to BOLD-100 as compared to the parental MM.1S cell line (BOLD-100 IC₅₀ of MM.1S=83 μM; MM.1S BR=23 UM); and (2B) the growth curves of the cell lines OPM2, OPM2 LR (lenalidomide resistant) and OPM2 PR (pomalidomide resistant) in the presence of increasing doses of BOLD-100 over 24 hours, demonstrating the higher sensitivity of the lenalidomide and pomalidomide resistant lines to BOLD-100 (BOLD-100 IC₅₀ of OPM2=120 UM; OPM2 PR=51 UM).

FIG. 3 includes a series of 12 paired bar graphs and scatter plots (FIGS. 3A to 3L), illustrating that BOLD-100 overcomes PI resistance in vitro. In FIGS. 3A to 3L, the bar graphs show percentage survival normalized to vehicle control for the indicated treatments. In FIGS. 3A to 3L the scatter plots show the corresponding combination index and fraction effect size for the treatments, with dotted lines indicating regions from top to bottom respectively of antagonism, additive effect and synergism. In FIG. 3M, bar graphs illustrate the same data as shown in other panels of FIG. 3, in which all cells in each data set are treated with 10 nM of bortezomib (left hand bar graph) or 10 nM carfilzomib (right hand bar graph).

FIG. 4 includes two paired bar graphs and photographs, illustrating the efficacy of BOLD-100 combined with bortezomib (Btz) in mediating a strong cytotoxic effect in both PI sensitive (MM.1S, FIG. 4A) and resistant (MM.1 BR, FIG. 4B) cell lines in a clonogenic assay.

FIG. 5 is a line graph illustrating that BOLD-100 negatively impacts the viability of three MM.1S cell lines, but has minimal impact on bone marrow cell line HS-5.

### DETAILED DESCRIPTION

As disclosed herein, BOLD-100 in combination with a PI is capable of mediating an additive therapeutic effect in PI sensitive cancer cells, and overcomes resistance in PI resistant cancer cells. Accordingly, when the therapeutic efficacy of a PI is reduced, typically due to acquired drug resistance from prolonged exposure, adding BOLD-100 to a treatment regimen restores efficacy of the PI. In addition, the combined use of BOLD-100 and PIs facilitates enduring PI therapy, in the sense that the additive combined use of BOLD-100 and a PI is further supplemented by a synergistic effect against any PI resistant cells that arise in the course of treatment. In this way, BOLD-100 may be used so as to inhibit the progression of a PI sensitive disease to PI resistance. These forms of enduring PI combination therapy with BOLD-100 may accordingly have particular utility in cancers or lymphoproliferative disorders that are susceptible to developing resistance to treatment with a PI. This is for example the case in MM, particularly in treating recurring/refractory MM or in preventing progression of MM to RRMM.

Aspects of the present invention involve methods for preparing drug products containing the sodium salt of trans-[tetrachlorobis(1H-indazole)ruthenate(III)](i.e. BOLD-100), as described below.

One aspect of the current invention provides a method for preparing a sterile, lyophilized drug product containing sodium trans-[tetrachlorobis(1H-indazole)ruthenate(III)]. This formulation would be suitable for administration to a patient. The formulation is comprised of sodium trans-[tetrachlorobis(1H-indazole)ruthenate(III)], a pH buffer, and a cryoprotective agent. The general method for providing said formulation comprises the steps of preparing aqueous buffer solution, preparing aqueous cryoprotectant solution, dissolution of sodium trans-[tetrachlorobis(1H-indazole)ruthenate(III)] in the buffer solution, addition of the cryoprotectant solution, sterile filtration (e.g. aseptic filtration), filling of vials under sterile conditions, and lyophilization under sterile conditions. Suitable buffers include, but are not limited to: citrate, TRIS, acetate, EDTA, HEPES, tricine, and imidazole. The use of a phosphate buffer is possible but is not preferred. A preferred aspect of the present invention is the use of a citric acid/sodium citrate buffer. Suitable cryoprotective agents include, but are not limited to: sugars, monosaccarides, disaccharides, polyalcohols, mannitol, sorbitol, sucrose, trehalose, dextran, and dextrose. A preferred aspect of the present invention is the use of mannitol as the cyroprotecive agent.

As described above, herein, sodium trans-[tetrachlorobis(1H-indazole)ruthenate(III)] can degrade in water to Compound A (Scheme II). One skilled in the art will recognize that limiting this degradation reaction would be advantageous to obtaining the highest purity product. It was found that cooling the sodium trans-[tetrachlorobis(1H-indazole)ruthenate(III)] solution during the formulation process was found to greatly reduce the amount of Compound A present in the lyophilized product. In one aspect of the invention, the sodium trans-[tetrachlorobis(1H-indazole) ruthenate (III)] solution is cooled to 4° C. during the formulation process. In another aspect of the invention, the sodium trans-[tetrachlorobis(1H-indazole)ruthenate(III)] solution is cooled to 2-8° C. during the formulation process. In another aspect of the invention, the sodium trans-[tetrachlorobis(1H-indazole)ruthenate(III)] solution is cooled to 2-15° C. during the formulation process.

One embodiment of the present invention provides a composition comprising sodium trans-[tetrachlorobis(1H-indazole)ruthenate(III)], a suitable buffer, and mannitol. In some embodiments, a suitable buffer comprises a citrate buffer. For instance, in some embodiments, a citrate buffer comprises sodium citrate and citric acid. An alternative embodiment of the present invention provides a composition comprising sodium trans-[tetrachlorobis(1H-indazole)ruthenate(III)], sodium citrate, citric acid, and mannitol. An alternative embodiment of the present invention provides a composition comprising sodium trans-[tetrachlorobis(1H-indazole)ruthenate(III)], sodium citrate, citric acid, mannitol, and mer, trans-[RuIIICl₃(Hind)₂(H₂O)]. An alternative embodiment of the present invention provides a composition comprising sodium trans-[tetrachlorobis(1H-indazole)ruthenate(III)], sodium citrate, citric acid, mannitol, mer, trans-[RuIIICl₃(Hind)₂(H₂O)], and a cesium salt. An alternative embodiment of the present invention provides a composition comprising sodium trans-[tetrachlorobis(1H-indazole)ruthenate(III)], sodium citrate, citric acid, and mannitol, wherein the sodium trans-[tetrachlorobis(1H-indazole)ruthenate(III)] is amorphous. An alternative embodiment of the present invention provides a composition comprising sodium trans-[tetrachlorobis(1H-indazole)ruthenate(III)], sodium citrate, citric acid, mannitol, and mer, trans-[RuIIICl₃(Hind)₂(H₂O)], wherein the sodium trans-[tetrachlorobis(1H-indazole)ruthenate(III)] is amorphous. An alternative embodiment of the present invention provides a composition comprising sodium trans-[tetrachlorobis(1H-indazole)ruthenate(III)], sodium citrate, citric acid, mannitol, mer, trans-[RuIIICl₃(Hind)₂(H₂O)], and a cesium salt, wherein the sodium trans-[tetrachlorobis(1H-indazole)ruthenate(III)] is amorphous. An alternative embodiment of the present invention provides a composition comprising sodium trans-[tetrachlorobis(1H-indazole) ruthenate (III)], sodium citrate, citric acid, mannitol, mer, trans-[RullICI3 (Hind)₂ (H₂O)], and a cesium salt;
wherein:
mer, trans-[Ru^{II} Cl₃(Hind)₂(H₂O)] is between about 0.01 and about 0.4 weight percent of the composition,
and cesium is between about 0.00001 and about 0.01 weight percent of the composition.

An alternative embodiment of the present invention provides a composition comprising sodium trans-[tetrachlorobis(1H-indazole)ruthenate(III)], sodium citrate, citric acid, mannitol, mer, trans-[Ru^{II}Cl₃(Hind)₂(H₂O)], and a cesium salt;
wherein:
mer, trans-[Ru^{II}Cl₃(Hind)₂(H₂O)] is between about 0.01 and about 0.4 weight percent of the composition,
and cesium is between about 0.00001 and about 0.01 weight percent of the composition.

An alternative embodiment of the present invention provides a composition comprising sodium trans-[tetrachlorobis(1H-indazole)ruthenate(III)], sodium citrate, citric acid, mannitol, mer, trans-[Ru^{II}Cl₃(Hind)₂(H₂O)], and a cesium salt;
wherein:
mer, trans-[Ru^{II}Cl₃(Hind)₂(H₂O)] is between about 0.01 and about 0.2 weight percent of the composition,
and cesium is between about 0.00001 and about 0.01 weight percent of the composition.

An alternative embodiment of the present invention provides a composition comprising sodium trans-[tetrachlorobis(1H-indazole)ruthenate(III)], mer, trans-[Ru^{II}Cl₃(Hind)₂(H₂O)], and a cesium salt;
wherein:
mer, trans-[Ru^{III}Cl₃(Hind)₂(H₂O)] is between about 0.01 and about 0.40 weight percent of the composition,
and cesium is between about 0.00001 and about 0.01 weight percent of the composition.

An alternative embodiment of the present invention provides a composition comprising sodium trans-[tetrachlorobis(1H-indazole)ruthenate(III)], mer, trans-[Ru^{III}Cl₃(Hind)₂(H₂O)], and a cesium salt;
wherein:
the composition is a lyophilized powder, mer, trans-[Ru^{III}Cl₃(Hind)₂(H₂O)] is between about 0.01 and about 0.40 weight percent of the composition,
and cesium is between about 0.00001 and about 0.01 weight percent of the composition.

An alternative embodiment of the present invention provides a composition comprising sodium trans-[tetrachlorobis(1H-indazole)ruthenate(III)], sodium citrate, citric acid, mannitol, mer, trans-[Ru^{II}Cl₃(Hind)₂(H₂O)], and a cesium salt;
wherein:
the composition is a lyophilized powder, mer, trans-[Ru^{II}Cl₃(Hind)₂(H₂O)] is between about 0.01 and about 0.3 weight percent of the composition,
and cesium is between about 0.00001 and about 0.1 weight percent of the composition.

An alternative embodiment of the present invention provides a composition comprising sodium trans-[tetrachlorobis(1H-indazole)ruthenate(III)], sodium citrate, citric acid, mannitol, mer, trans-[Ru^{II}Cl₃(Hind)₂(H₂O)], and a cesium salt;
wherein:
mer, trans-[Ru^{III}Cl₃(Hind)₂(H₂O)] is between about 0.01 and about 0.3 weight percent of the composition,
and cesium is between about 0.00001 and about 0.1 weight percent of the composition.

An alternative embodiment of the present invention provides a composition comprising sodium trans-[tetrachlorobis(1H-indazole)ruthenate(III)], sodium citrate, citric acid, mannitol, mer, trans-[RuIICl₃(Hind)₂(H₂O)], and a cesium salt;
wherein:
the composition is a lyophilized powder,
sodium trans-[tetrachlorobis(1H-indazole)ruthenate(III)] is about 11.5 to about 14.0 weight percent of the composition,
citric acid is about 43.9 to about 53.7 weight percent of the composition,
sodium citrate is about 25.7 to about 23.1 weight percent of the composition,
mannitol is about 11.5 to about 14.0 weight percent of the composition,
mer, trans-[Ru^{III}Cl₃(Hind)₂(H₂O)] is about 0.01 and about 0.3 weight percent of the composition,
and cesium is between about 0.00001 and about 0.1 weight percent of the composition.

An alternative embodiment of the present invention provides a composition comprising sodium trans-[tetrachlorobis(1H-indazole)ruthenate(III)], sodium citrate, citric acid, mannitol, mer, trans-[RuIICl₃(Hind)₂ (H₂O)], and a cesium salt;
wherein:
the composition is a lyophilized powder,
sodium trans-[tetrachlorobis(1H-indazole)ruthenate(III)] is about 10.2 to about 15.3 weight percent of the composition,
citric acid is about 39.0 to about 58.5 weight percent of the composition,
sodium citrate is about 20.5 to about 30.8 weight percent of the composition,
mannitol is about 10.2 to about 15.3 weight percent of the composition,
mer, trans-[Ru^{III}Cl₃(Hind)₂(H₂O)] is about 0.01 and about 0.3 weight percent of the composition,
and cesium is between about 0.00001 and about 0.1 weight percent of the composition.

An alternative embodiment of the present invention provides a composition comprising sodium trans-[tetrachlorobis(1H-indazole)ruthenate(III)], sodium citrate, citric acid, mannitol, mer, trans-[Ru^{II}Cl₃(Hind)₂(H₂O)], and a cesium salt;
wherein:
the composition is a lyophilized powder,
sodium trans-[tetrachlorobis(1H-indazole)ruthenate(III)] is about 10.2 to about 15.3 weight percent of the composition,
mer, trans-[Ru^{III}Cl₃(Hind)₂(H₂O)] is about 0.01 and about 0.3 weight percent composition,
and cesium is between about 0.00001 and about 0.1 weight percent of the composition.

An alternative embodiment of the present invention provides a composition comprising sodium trans-[tetrachlorobis(1H-indazole)ruthenate(III)], mannitol, citric acid, and sodium citrate;
wherein:
sodium trans-[tetrachlorobis(1H-indazole)ruthenate(III)] is about 49.86 weight percent of the composition,
mannitol is about 49.86 weight percent of the composition,
citric acid is about 0.187 weight percent of the composition,
and sodium citrate is about 0.093 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, and sodium citrate;
wherein:
sodium trans-[tetrachlorobis(1H-indazole)ruthenate(III)] is about 40 to about 60 weight percent of the composition,
mannitol is about 40 to about 60 weight percent of the composition,
citric acid is about 0.01 to about 0.5 weight percent of the composition,
and sodium citrate is about 0.001 to about 0.25 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, and sodium citrate;
wherein:
sodium trans-[tetrachlorobis(1H-indazole)ruthenate(III)] is about 30 to about 70 weight percent of the composition,
mannitol is about 30 to about 70 weight percent of the composition,
citric acid is about 0.001 to about 1 weight percent of the composition,
and sodium citrate is about 0.0001 to about 1 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, and Ru^{II}Cl₃(Hind)₂(H₂O);
wherein:
sodium trans-[tetrachlorobis(1H-indazole)ruthenate(III)] is about 49.86 weight percent of the composition,
mannitol is about 49.86 weight percent of the composition,
citric acid is about 0.187 weight percent of the composition,
sodium citrate is about 0.093 weight percentage of the composition,
and Ru^{II}Cl₃(Hind)₂(H₂O) is not more than 0.5 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, and Ru^{III}Cl₃(Hind)₂ (H₂O);
wherein:
sodium trans-[tetrachlorobis(1H-indazole)ruthenate(III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
and Ru^{III}Cl₃(Hind)₂ (H₂O) is about 0 to about 0.5 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{III}Cl₃(Hind)₂(H₂O), and cesium;
wherein:
sodium trans-[tetrachlorobis(1H-indazole)ruthenate(III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
Ru^{III}Cl₃(Hind)₂(H₂O) is not more than 0.5 weight percentage of the composition,
and cesium is not more than 0.25 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, and cesium;
wherein:
sodium trans-[tetrachlorobis(1H-indazole)ruthenate(III)] is about 49.61 weight percent of the composition,
mannitol is about 49.86 weight percent of the composition,
citric acid is about 0.187 weight percent of the composition,
sodium citrate is about 0.093 weight percentage of the composition
and cesium is about 0.25 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, and cesium;
wherein:
sodium trans-[tetrachlorobis(1H-indazole)ruthenate(III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
and cesium is about 0.1 to about 0.5 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, and cesium;
wherein:
sodium trans-[tetrachlorobis(1H-indazole)ruthenate(III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
and cesium is about 0.01 to about 1 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{II}Cl₃(Hind)₂(H₂O), Ru^{II}Cl₃(Hind)₂(CH₃CN), Ru^{II}Cl₃(Hind) (HN═C (Me) ind), and cesium;
wherein:
sodium trans-[tetrachlorobis(1H-indazole)ruthenate(III)] about 46.61 weight percent of the composition,
mannitol is about 49.86 weight percent of the composition,
citric acid is about 0.187 weight percent of the composition,
sodium citrate is about 0.093 weight percentage of the composition,
Ru^{II}Cl₃(Hind)₂(H₂O) is not more than 0.5 weight percentage of the composition,
Ru^{III}Cl₃(Hind)₂(CH₃CN) is not more than 1.25 weight percentage of the composition,
Ru^{II}Cl₃(Hind) (HN═C (Me) ind) is not more than 1.0 weight percentage of the composition,
and cesium is not more than 0.25 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{III}Cl₃(Hind)₂(H₂O), Ru^{III}Cl₃(Hind)₂(CH₃CN), Ru^{II}Cl₃(Hind) (HN═C (Me) ind), and cesium;
wherein:
sodium trans-[tetrachlorobis(1H-indazole)ruthenate(III)] about between 46.61 weight percent of the composition,
mannitol is about 49.86 weight percent of the composition,
citric acid is about 0.187 weight percent of the composition,
sodium citrate is about 0.093 weight percentage of the composition,
Ru^{II}Cl₃(Hind)₂(H₂O) is not more than 0.5 weight percentage of the composition,
Ru^{III}Cl₃(Hind)₂(CH₃CN) is not more than 1.25 weight percentage of the composition,
Ru^{II}Cl₃(Hind) (HN═C (Me) ind) is not more than 1.0 weight percentage of the composition,
and cesium is not more than 0.25 weight percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{II}Cl₃(Hind)₂(H₂O), Ru^{II}Cl₃(Hind)₂(CH₃CN), Ru^{II}Cl₃(Hind) (HN═C (Me) ind), and cesium;
wherein:
sodium trans-[tetrachlorobis(1H-indazole)ruthenate(III)] is about 40 to about 60 weight percent of the composition,
mannitol is about 40 to about 60 weight percent of the composition,
citric acid is about 0.01 to about 0.5 weight percent of the composition,
sodium citrate is about 0.001 to about 0.25 weight percentage of the composition,
Ru^{II}Cl₃(Hind)₂(H₂O) is not more than about 0.5 weight percentage of the composition,
Ru^{III}Cl₃(Hind)₂(CH₃CN) is not more than about 1.25 weight percentage of the composition,
Ru^{II}Cl₃(Hind) (HN═C (Me) ind) is not more than about 1.0 weight percentage of the composition,
and cesium is not more than 0.25 percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{II}Cl₃(Hind)₂(H₂O), Ru^{II}Cl₃(Hind)₂(CH₃CN), Ru^{II}Cl₃(Hind) (HN═C (Me) ind), and cesium;
wherein:
sodium trans-[tetrachlorobis(1H-indazole)ruthenate(III)] is about 30 to about 70 weight percent of the composition,
mannitol is about 30 to about 70 weight percent of the composition,
citric acid is about 0.001 to about 1 weight percent of the composition,
sodium citrate is about 0.0001 to about 1 weight percentage of the composition,
Ru^{III}Cl₃(Hind)₂(H₂O) is not more than about 0.5 weight percentage of the composition,
Ru^{II}Cl₃(Hind)₂(CH₃CN) is not more than about 1.25 weight percentage of the composition,
Ru^{II}Cl₃(Hind) (HN═C (Me) ind) is not more than about 1.0 weight percentage of the composition,
and cesium is not more than 0.25 percentage of the composition. In some such embodiments, the composition is a lyophilized powder.

An alternative embodiment of the present invention provides a composition comprising sodium trans-[tetrachlorobis(1H-indazole)ruthenate(III)], mannitol, citric acid, sodium citrate, Ru^{II}Cl₃(Hind)₂(H₂O), Ru^{III}Cl₃(Hind)₂(CH₃CN), Ru^{II}Cl₃(Hind) (HN═C (Me) ind), and cesium;
wherein:
sodium trans-[tetrachlorobis(1H-indazole)ruthenate(III)] is about 20 to about 80 weight percent of the composition,
mannitol is about 20 to about 80 weight percent of the composition,
citric acid is about 0.0001 to about 5 weight percent of the composition,
sodium citrate is about 0.00001 to about 5 weight percentage of the composition,
Ru^{II}Cl₃(Hind)₂(H₂O) is not more than about 0.5 weight percentage of the composition,
Ru^{II}Cl₃(Hind)₂(CH₃CN) is not more than about 1.25 weight percentage of the composition,
Ru^{II}Cl₃(Hind) (HN═C (Me) ind) is not more than about 1.0 weight percentage of the composition,
and cesium is not more than 0.25 percentage of the composition. In some such embodiments, the composition is a lyophilized powder.

In some embodiments, the present invention provides a unit dosage form comprising a formulation or composition described herein. The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Compositions of the present invention can be provided as a unit dosage form. In some embodiments, a vial comprising sodium trans-[tetrachlorobis(1H-indazole)ruthenate(III)], mannitol, citric acid, sodium citrate is a unit dosage form.

In some embodiments, the present invention a vial comprising sodium trans-[tetrachlorobis(1H-indazole)ruthenate(III)], mannitol, citric acid, sodium citrate, and cesium is a unit dosage form.

In some embodiments, the present invention a vial comprising sodium trans-[tetrachlorobis(1H-indazole)ruthenate(III)], mannitol, citric acid, sodium citrate, Ru^{III}Cl₃(Hind)₂(H₂O), Ru^{II}Cl₃(Hind)₂(CH₃CN), Ru^{II}Cl₃(Hind) (HN═C (Me) ind), and cesium is a unit dosage form.

Still further encompassed by the invention are pharmaceutical packs and/or kits comprising compositions described herein, or a unit dosage form comprising a provided composition, and a container (e.g., a foil or plastic package, or other suitable container). Optionally instructions for use are additionally provided in such kits.

In some embodiments, the present invention can be provided as a unit dosage form. Indeed, a vial comprising sodium trans-[tetrachlorobis(1H-indazole)ruthenate(III)], mannitol, citric acid, sodium citrate is a unit dosage form depicted in Table 3

TABLE 3
Pharmaceutical Components

| | | Weight | Amount/ | |
|---|---|---|---|---|
| Component | Function | % | vial | |
| sodium trans-[tetrachlorobis(1H- | Active | 47.5 | 100 | mg |
| indazole)ruthenate (III)] | | | | |
| Mannitol | Cryoprotectant | 47.5 | 100 | mg |
| Citric Acid | Buffer component | 3.37 | 7.1 | mg |
| Sodium citrate | Buffer component | 1.63 | 3.4 | mg |

In some embodiments, the pharmaceutical components described in Table 3 further comprise cesium;
wherein:
cesium is not more than 0.25 weight percentage of the composition.

In some embodiments, the pharmaceutical components described in Table 3 further comprise cesium, Ru^{II}Cl₃(Hind)₂(H₂O), Ru^{II}Cl₃(Hind)₂(CH₃CN), and Ru^{II}Cl₃(Hind) (HN═C (Me) ind);
wherein:
cesium is not more than about 0.25 weight percentage of the composition,
Ru^{II}Cl₃(Hind)₂(H₂O) is not more than about 0.5 weight percentage of the composition,
Ru^{II}Cl₃(Hind)₂(CH₃CN) is not more than about 1.25 weight percentage of the composition,
and Ru^{II}Cl₃(Hind) (HN═C (Me) ind) is not more than about 1.0 weight percentage of the composition.

In some embodiments, the pharmaceutical composition is selected from those in Table 4:

TABLE 4
Pharmaceutical Component Ranges

| | | Weight % | | |
|---|---|---|---|---|
| Component | Function | Range | Amount / vial | |
| sodium trans-[tetrachlorobis(1H- | Active | 42.75-52.25 | 90-110 | mg |
| indazole)ruthenate (III)] | | | | |
| Mannitol | Cryoprotectant | 42.75-52.25 | 90-110 | mg |
| Citric Acid | Buffer component | 3.033-3.707 | 6.39-7.81 | mg |
| Sodium citrate | Buffer component | 1.467-1.793 | 3.06-3.74 | mg |

In some embodiments, the pharmaceutical components described in Table 4 further comprise cesium;
wherein:
cesium is not more than 0.25 weight percentage of the composition.

In some embodiments, the pharmaceutical components described in Table 4 further comprise cesium, Ru^{II}Cl₃(Hind)₂(H₂O), Ru^{II}Cl₃(Hind)₂(CH₃CN), and Ru^{II}Cl₃ (Hind) (HN═C (Me) ind);
wherein:
cesium is not more than about 0.25 weight percentage of the composition,
Ru^{II}Cl₃(Hind)₂(H₂O) is not more than about 0.5 weight percentage of the composition,
Ru^{II}Cl₃(Hind)₂(CH₃CN) is not more than about 1.25 weight percentage of the composition,
and Ru^{III}Cl₃(Hind) (HN═C (Me) ind) is not more than about 1.0 weight percentage of the composition.

In some embodiments, the present invention can be provided as a unit dosage form. Indeed, a vial comprising sodium trans-[tetrachlorobis(1H-indazole)ruthenate(III)], mannitol, citric acid, sodium citrate is a unit dosage form depicted in Table 5:

TABLE 5
Pharmaceutical Components

| Component | Function | Weight % | Amount / vial | |
|---|---|---|---|---|
| sodium trans-[tetrachlorobis(1H- | Active | 49.86 | 300 | mg |
| indazole)ruthenate (III)] | | | | |
| Mannitol | Cryoprotectant | 49.86 | 300 | mg |
| Citric Acid | Buffer component | 0.188 | 1.13 | mg |
| Sodium citrate | Buffer component | 0.092 | 0.55 | mg |

In some embodiments, the pharmaceutical components described in Table 5 further comprise cesium;
wherein:
cesium is not more than 0.25 weight percentage of the composition.

In some embodiments, the pharmaceutical components described in Table 5 further comprise cesium, Ru^{II}Cl₃(Hind)₂(H₂O), Ru^{III}Cl₃(Hind)₂(CH₃CN), and Ru^{II}Cl₃(Hind) (HN═C (Me) ind);
wherein:
cesium is not more than about 0.25 weight percentage of the composition,
Ru^{II}Cl₃(Hind)₂(H₂O) is not more than about 0.5 weight percentage of the composition,
Ru^{II}Cl₃(Hind)₂(CH₃CN) is not more than about 1.25 weight percentage of the composition,
and Ru^{II}Cl₃(Hind) (HN═C (Me) ind) is not more than about 1.0 weight percentage of the composition.

In some embodiments, the pharmaceutical composition is selected from those in Table 6:

TABLE 6
Pharmaceutical Components

| | | Weight % | | |
|---|---|---|---|---|
| Component | Function | Range | Amount / vial | |
| sodium trans-[tetrachlorobis(1H- | Active | 44.87-54.85 | 270-330 | mg |
| indazole)ruthenate (III)] | | | | |
| Mannitol | Cryoprotectant | 44.87-54.85 | 270-330 | mg |
| Citric Acid | Buffer component | 0.169-0.207 | 1.02-1.24 | mg |
| Sodium citrate | Buffer component | 0.0828-0.1012 | 0.495-0.605 | mg |

In some embodiments, the pharmaceutical components described in Table 6 further comprise cesium;
wherein:
cesium is not more than 0.25 weight percentage of the composition.

In some embodiments, the pharmaceutical components described in Table 6 further comprise cesium, Ru^{II}Cl₃(Hind)₂(H₂O), Ru^{III}Cl₃(Hind)₂(CH₃CN), and Ru^{II}Cl₃(Hind) (HN═C (Me) ind);
wherein:
cesium is not more than about 0.25 weight percentage of the composition,
Ru^{II}Cl₃(Hind)₂(H₂O) is not more than about 0.5 weight percentage of the composition,
Ru^{II}Cl₃(Hind)₂(CH₃CN) is not more than about 1.25 weight percentage of the composition,
and Ru^{III}Cl₃(Hind) (HN═C (Me) ind) is not more than about 1.0 weight percentage of the composition.

In some embodiments, the pharmaceutical components are as described in any of Tables 3-6, and further comprise cesium. In some embodiments, cesium is present in an amount of about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0 weight percentage of the composition.

In some embodiments, the effective amount of BOLD-100 may be effective to reduce the amount of GRP78 in cells following administration of BOLD-100, either alone or in combination, for example in combination with a PI.

In certain embodiments, BOLD-100 is administered after the PI, for example at least 1, 2, 3, 4, 5, 6 or 7 days after, or between 1 and seven days after the PI. Alternatively, BOLD-100 may be administered at least approximately simultaneously with the PI, for example within 1, 2, 3, 4, 5 or 6 hours of the PI, or within 20, 21, 22, 23, 24, 25, 26, 27 or 28 hours of each other. Alternatively, BOLD-100 may be administered before the PI, such as at least about 8, 9, 10, 11, 12, 13, 14, 15 or 16 hours before the PI.

In certain embodiments, treatment with BOLD-100 may be part of a first line or induction treatment, alternatively BOLD-100 may be used in the treatment of relapsing or refractory disease. In MM or RRMM for example, BOLD-100 may be combined with one or more of a PI (such as bortezomib, carfilzomib or ixazomib), lenalidomide, pomalidomide, dexamethasone, prednisone, cyclophosphamide, thalidomide, daratumumab, elotuzumab, isatuximab, selinexor, melphalan, cyclophosphamide, doxorubicin, liposomal doxorubicin and panobinostat. MM treatment with BOLD-100 may for example be prior to autologous stem cell transplant.

A titratable dosage may for example be adapted to allow a patient to take the medication in doses smaller than the unit dose, wherein a “unit dose” is defined as the maximum dose of medication that can be taken at any one time or within a specific dosage period. Titration of doses will allow different patients to incrementally increase the dose until they feel that the medication is efficacious, as not all patients will require the same dose to achieve the same benefits. A person with a larger build or faster metabolism may require larger doses to achieve the same effect as another with a smaller build or slower metabolism. Therefore, a titratable dosage has advantages over a standard dosage form.

In select embodiments, formulations may be adapted to be delivered in such a way as to target one or more of the following: sublingual, buccal, oral, rectal, nasal, parenteral and via the pulmonary system. Formulations may for example be in one or more of the following forms: gel, gel spray, tablet, liquid, capsule, by injection, or for vaporization.

Conventional pharmaceutical practice may be employed to provide suitable formulations or compositions to administer the formulations to subjects. Routes of administration may for example include, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Methods well known in the art for making formulations are found in, for example, “Remington: The Science and Practice of Pharmacy” (21st edition), ed. David Troy, 2006, Lippincott Williams & Wilkins. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Pharmaceutical composition of the invention are formulated so as to allow the active ingredients contained therein to be bioavailable upon administration of the composition to a patient. Compositions that will be administered to a patient may take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a formulation of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid formulation for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent. When the composition is in the form of a capsule, e.g., a gelatin capsule, it may contain, in addition to materials of the above type, a liquid carrier such as polyethylene glycol or a fatty oil.

The formulation may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, preferred compositions contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical formulations of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical formulation may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The formulation may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The formulation may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical formulation may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

Some biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

The present invention also provides kits that contain a pharmaceutical formulation, together with instructions for the use of the formulation. Preferably, a commercial package will contain one or more unit doses of the formulation. Formulations which are light and/or air sensitive may require special packaging and/or formulation. For example, packaging may be used which is opaque to light, and/or sealed from contact with ambient air, and/or formulated with suitable coatings or excipients.

The formulations of the invention can be provided alone or in combination with other compounds (for example, small molecules, nucleic acid molecules, peptides, or peptide analogues), in the presence of a carrier or any pharmaceutically or biologically acceptable carrier. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration. Pharmaceutically acceptable carriers generally include sterile aqueous solutions or dispersions and sterile powders. Supplementary active compounds can also be incorporated into the formulations.

An “effective amount” of a formulation according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a formulation may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the formulation or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

In therapeutic applications, synergy between active ingredients occurs when an observed combined therapeutic effect is greater than the sum of therapeutic effects of individual active ingredients, or a new therapeutic effect is produced that the active ingredients could not produce alone. Accordingly, when components of a formulation are present in synergistically effective amounts, the formulation yields a therapeutic effect that is greater than would be achieved by the individual active ingredients administered alone at comparable dosages. In this context, the enhancement of therapeutic effect may take the form of increased efficacy or potency and/or decreased adverse effects. The synergistic effect may be mediated in whole or in part by the pharmacokinetics and/or pharmacodynamics of the active ingredients in a subject, so that the amount and proportion of the ingredients in the formulation may be synergistic in vivo. This in vivo synergy may be effected with a formulation that includes the active ingredients in amounts and proportions that are also synergistic in in vitro assays of efficacy. As used herein, the term “synergistically effective amounts” accordingly refers to amounts that are synergistic in vivo and/or in vitro.

Synergy may be numerically reflected in compound interactions that are calculated by multiple drug effect analysis and performed by the median equation principle according to the methodology described by Chou and Talalay using the Compusyn software, version 1.0 (see Chou T C. “Drug combination studies and their synergy quantification using the Chou-Talalay method.” Cancer Res. 2010 Jan. 15;70 (2): 440-6). The CI values indicate synergistic, additive or antagonistic behavior of the drug combination. Cl<1, =1, and >1 indicate synergism, additive effect and antagonism, respectively.

An alternative numeric quantification of synergy is often expressed as a fractional inhibitory concentration index (FICI), which represents the sum of the fractional inhibitory concentrations (FICs) of each drug tested, where the FIC is determined for each drug by dividing the minimum inhibitory concentration (MIC, the lowest concentration of the drug which prevents visible growth of the bacterium in a standard in vitro assay-standard colorometric assay based on resazurin) of each drug when used in combination by the MIC of each drug when used alone. In very general terms, a FICI lower or higher than 1 indicates positively correlated activity (at least additive synergy) or an absence of positive interactions, respectively. More definitively, synergy of two compounds may be conservatively defined as a FICI of ≤0.5 (see Odds, 2003; with additivity or additive synergy corresponding to a FICI of >0.5 to ≤1; no interaction (indifference) corresponding to a FICI of >1 to ≤4; and antagonism corresponding to a FICI of >>4). Synergy of three compounds has been defined as a FICI of ≤1.0. (Berenbaum, 1978; Yu et al., 1980).

### EXAMPLES

As illustrated in the following Examples, BOLD-100 reverses PI resistance.

### Example 1: Sensitivity to BOLD-100

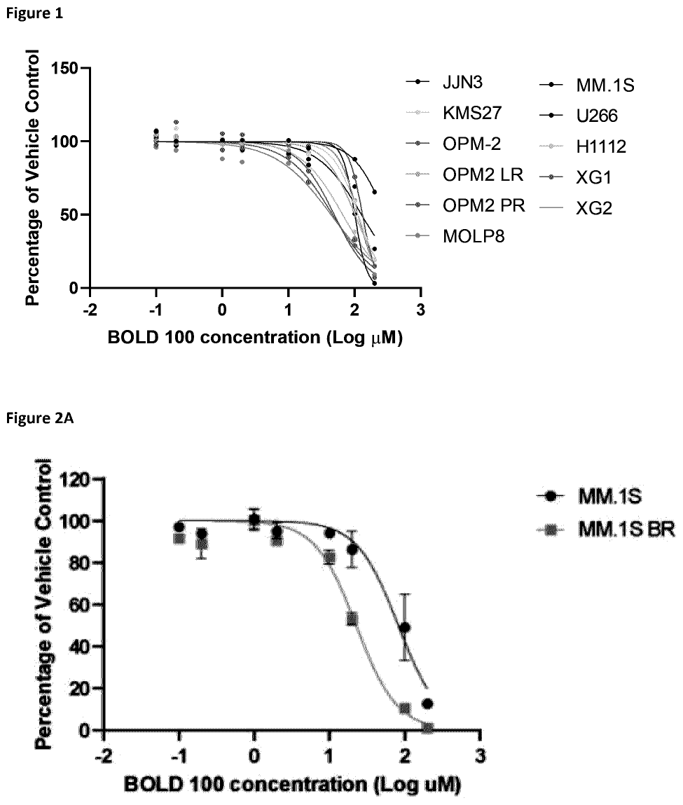

The anticancer activity of sodium trans-[tetrachloridobis(1H-indazole)ruthenate(III)](BOLD-100) was demonstrated by the inhibition of growth of various Multiple Myeloma (MM) cancer cell lines. Specifically, various MM cell lines were treated with BOLD-100 for 24 hours and subjected to a Cell Titer Glo assay which provides a metabolic read out that corresponds to the number of metabolically active and viable cells in culture. The luminescent readings obtained in the assay were used to measure the percentage of cell death following treatment with increasing doses of BOLD-100 for 24 hours. Each cell line was treated in triplicate and the average cell death was used to calculate the IC₅₀ or half-minimal inhibitory concentration dose in these cell lines using a nonlinear regression fit. FIG. 1 shows the growth curves of the different cell lines in presence of increasing doses of BOLD-100. Table 1 shows the IC₅₀ values of BOLD-100 in the respective cell lines.

TABLE 1

| Cell line | IC50 (μM) |
|---|---|
| JJN3 | Not reached |
| KMS27 | 128 |
| OPM2 | 130 |
| OPM2 LR | 108 |
| OPM2 PR | 51 |
| MOLP8 | 115 |
| MM.1S | 106 |
| H1112 | 72 |
| XG-1 | 49 |
| XG-2 | 54 |

### Example 2: Drug Resistant Cells Lines are More Sensitive to BOLD-100

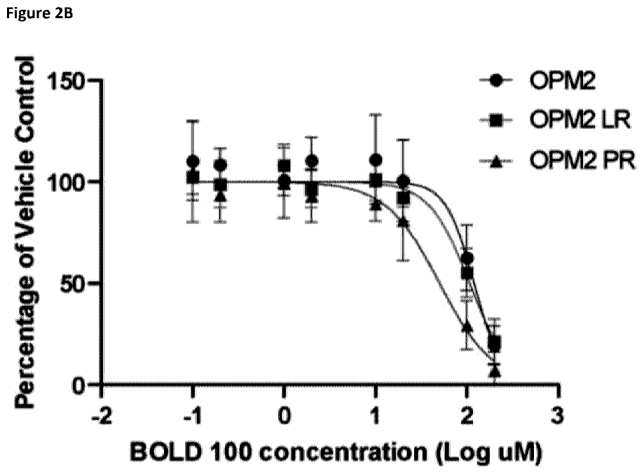

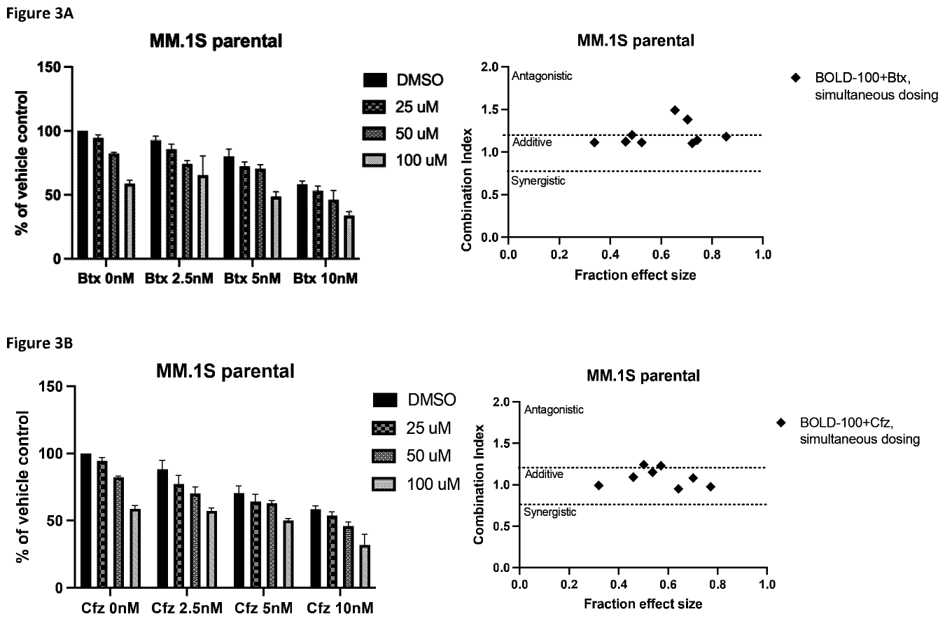

Proteasome inhibitors (PI) and immunomodulatory drugs (IMiD) are used as standards of care treatments in MM disease in the clinic. BOLD-100 was found to be more potent in MM cell lines that were developed to acquire resistance to each of these classes of drugs, as compared to the sensitive controls as demonstrated in in vitro CTG assays. Specifically, the MM.1S cell line was developed to acquire partial resistance to proteasome inhibitors bortezomib (MM.1S BR) and carfilzomib (MM.1S CR) by treating the MM.1S cell line with increasing doses of the drugs for about 6 months and the OPM2 cell line was developed to acquire partial resistance to IMiD lenalidomide (OPM2 LR) and pomalidomide (OPM2 PR) by treating the OPM2 cell line with increasing doses of the drugs for about 6 months. Once resistance was developed, the cells were tested for their capacity to grow independent of the presence of drugs, and all drug resistant cell lines were found to be able to grow in absence of bortezomib/carfilzomib and lenalidomide/pomalidomide, while maintaining their growth rates in absence of the drugs. FIG. 2A shows the growth curves of MM.1S and MM.1S BR in presence of increasing doses of BOLD-100 over 24 hours demonstrating the higher sensitivity of the PI-resistant line to BOLD-100 as compared to the parental MM.1S (BOLD-100 IC₅₀ of MM.1S=83 μM; MM.1S BR=23 μM). FIG. 2B shows the growth curves of the OPM2, OPM2 LR and OPM2 PR in presence of increasing doses of BOLD-100 over 24 hours demonstrating the higher sensitivity of the lenalidomide and pomalidomide resistant lines to BOLD-100 (BOLD-100 IC₅₀ of OPM2=120 UM; OPM2 PR=51 UM).

### Example 3: BOLD-100 Overcomes PI Resistance In Vitro

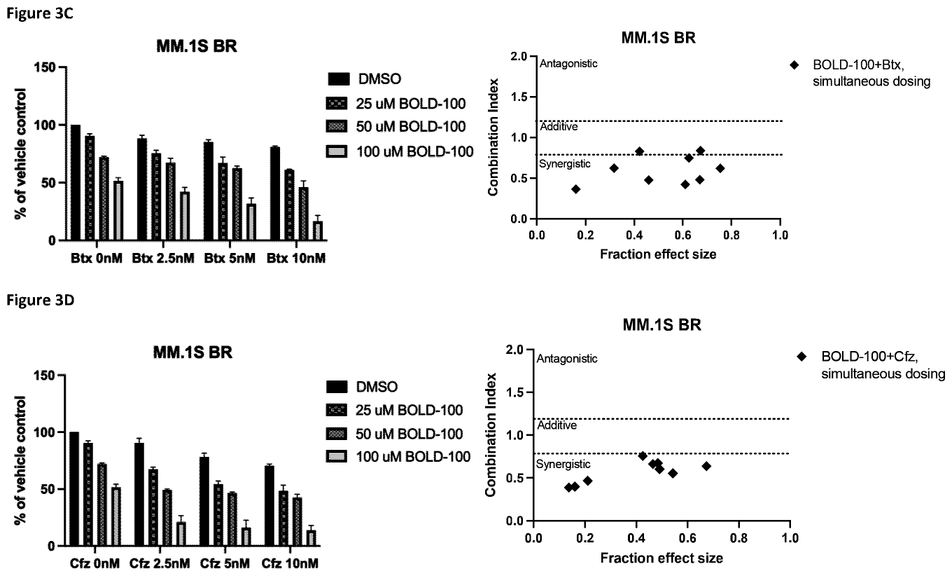

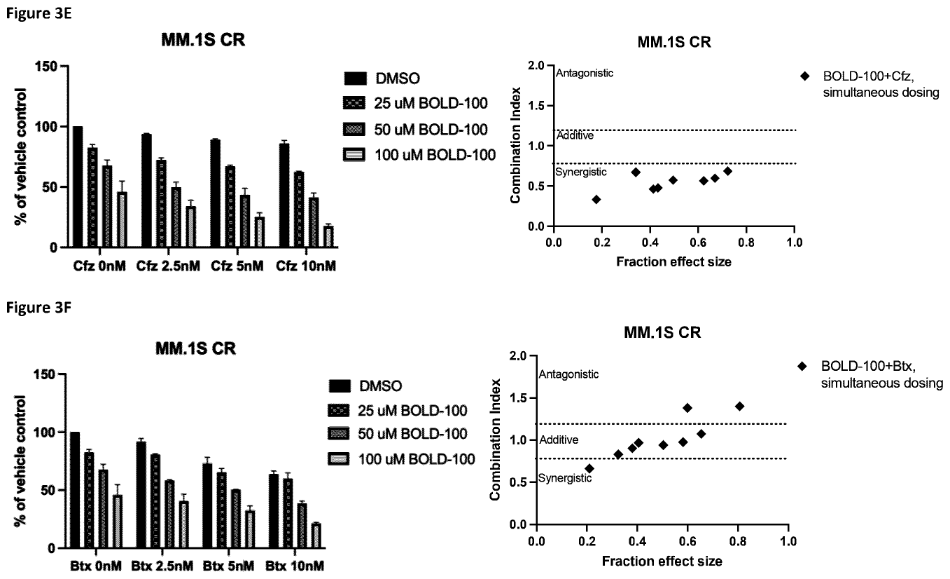

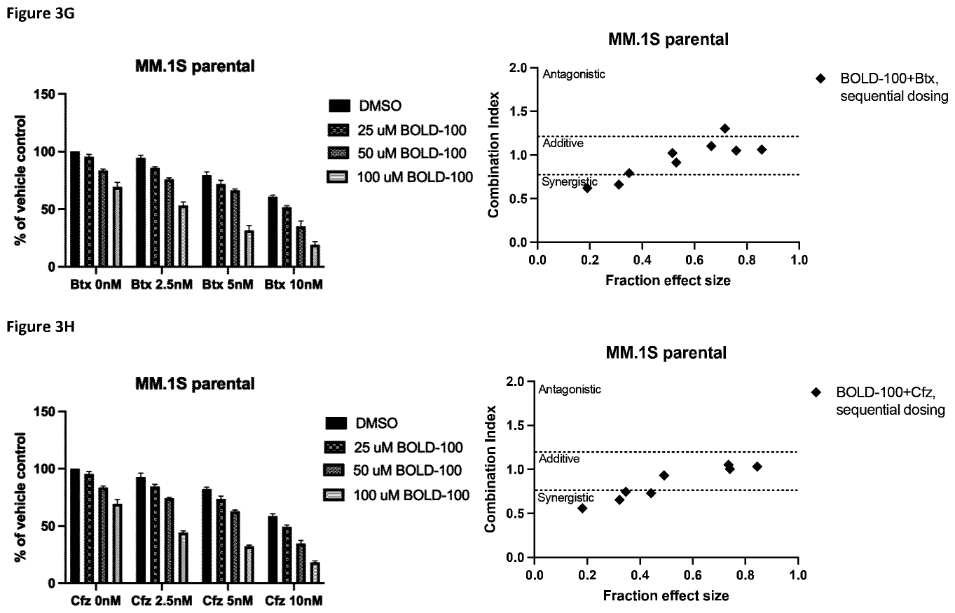

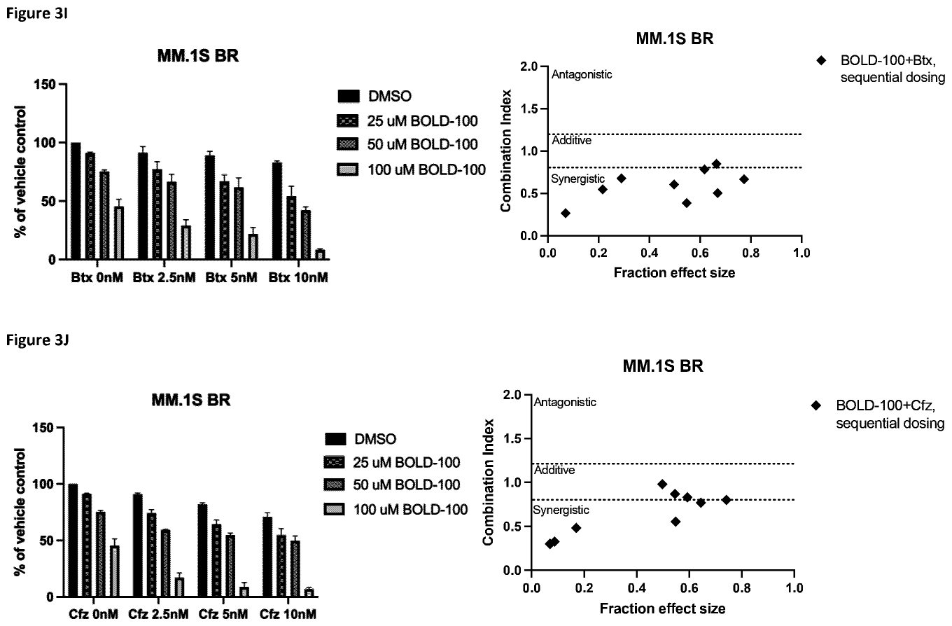

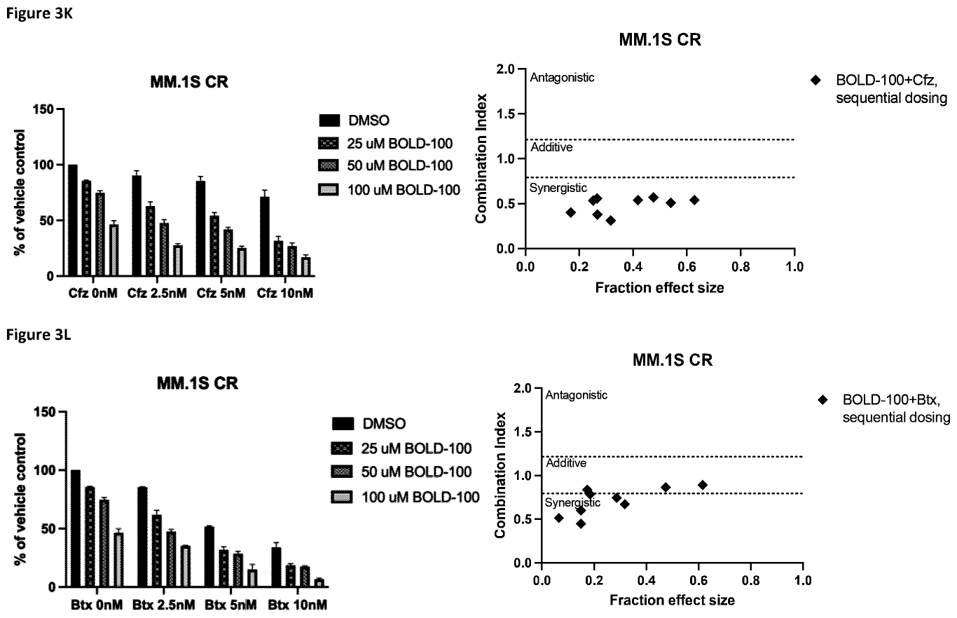

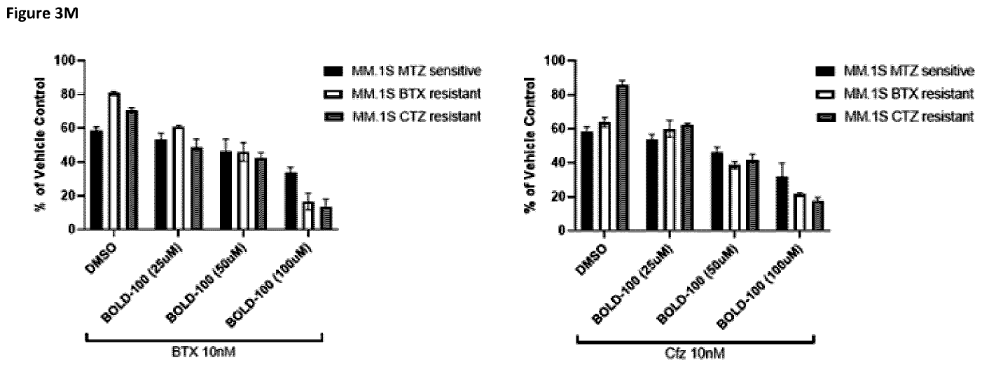

Cell lines were subjected to BOLD-100+PI treatment to illustrate how BOLD-100 was able to impact the viability of these resistance cell lines and overcome the resistance to PI. The PI sensitive and PI resistant cells were simultaneously treated with 3 increasing doses both drugs, namely BOLD-100 and the respective PI bortezomib (Btx) or carfilzomib (Cfz) for 24 hours and subjected to CTG assay to determine the viability (FIGS. 3A-3F) or sequentially treated with the drugs, where cells were first exposed to the proteasome inhibitor (bortezomib or carfilzomib) for 24 hours followed by addition of BOLD-100 for another 24 hours (FIGS. 3G-3L).

The percentage survival normalized to vehicle control in each cell line was represented as the fraction affected by the drug combination at the concentrations used (the “Fraction effect size”). Compound interactions were calculated as a Combination Index (CI) by multiple drug effect analysis, performed by the median equation principle according to the methodology described by Chou and Talalay using the Compusyn software, version 1.0 (see Chou T C. “Drug combination studies and their synergy quantification using the Chou-Talalay method.” Cancer Res. 2010 Jan. 15;70 (2): 440-6). The CI values are one way to indicate synergistic, additive or antagonistic behaviour of the drug combination. Using this method, as illustrated in the Figures, Cl<0.8, =0.8-1.2, and >1.2 indicate synergism, additive effect and antagonism, respectively. FIG. 3 represents data derived from the growth curves of the parental and resistant cells in presence of the drug combinations listed below, demonstrating decrease in survival with increasing concentration of both drugs and the respective CI vs Fraction effect size plots of each cell line showing synergism, additivity or antagonism:

### Bortezomib+BOLD-100

B100-25 μM/Btx 2.5 nM
B100-50 μM/Btx 2.5 nM
B100-100 μM/Btx 2.5 nM
B100-25 μM/Btx 5 nM
B100-50 μM/Btx 5 nM
B100-100 μM/Btx 5 nM
B100-25 μM/Btx 10 nM
B100-100 μM/Btx 10 nM
B100-100 μM/Btx 10 nM

### Carfilzomib+BOLD-100

B100-25 μM/Cfz 2.5 nM
B100-50 μM/Cfz 2.5 nM
B100-100 μM/Cfz 2.5 nM
B100-25 μM/Cfz 5 nM
B100-50 μM/Cfz 5 nM
B100-100 μM/Cfz 5 nM
B100-25 μM/Cfz 10 nM
B100-100 μM/Cfz 10 nM
B100-100 μM/Cfz 10 nM
(B100 is BOLD-100, Btx is bortezomib, Cfz is carfilzomib).

FIG. 3A and FIG. 3B show that the combination of BOLD-100+bortezomib or carfilzomib in the PI sensitive MM cell lines show an additive effect of the combination, while FIG. 3C and FIG. 3E show that BOLD-100 when combined with either bortezomib or carfilzomib in the matched resistant cell lines demonstrate a synergistic effect on cell viability. For the bortezomib resistant cell line the combination of BOLD-100 with carfilzomib as shown in FIG. 3D also shows synergistic impact on cell viability, indicating cross resistance, an effect not seen in carfilzomib resistant cell line when BOLD-100 is combined with bortezomib in FIG. 3F. The effect of simultaneous dosing shown in FIGS. 3A to 3F is evident also in the corresponding sequential dosing data shown in FIGS. 3G to 3L.

An alternative way of illustrating the effect shown in the FIG. 3 data is provided in FIG. 3L, in which all cells in each data set are treated with 10 nM of bortezomib (left hand bar graph) or 10 nM carfilzomib (right hand bar graph). As illustrated, when BOLD-100 was not added (DMSO columns) there is less response with resistant cell lines compared to sensitive cell lines. Importantly, as the concentration of BOLD-100 increases, the relative efficacy of treatment of sensitive vs resistant cells reverses. For example, at 100 μM BOLD-100 there is an approximate doubling of cell death in the resistant cell lines compared to the sensitive cell lines.

### Example 4: Efficacy of BOLD-100 in PI Resistant Cells Demonstrated in Clonogenic Assay

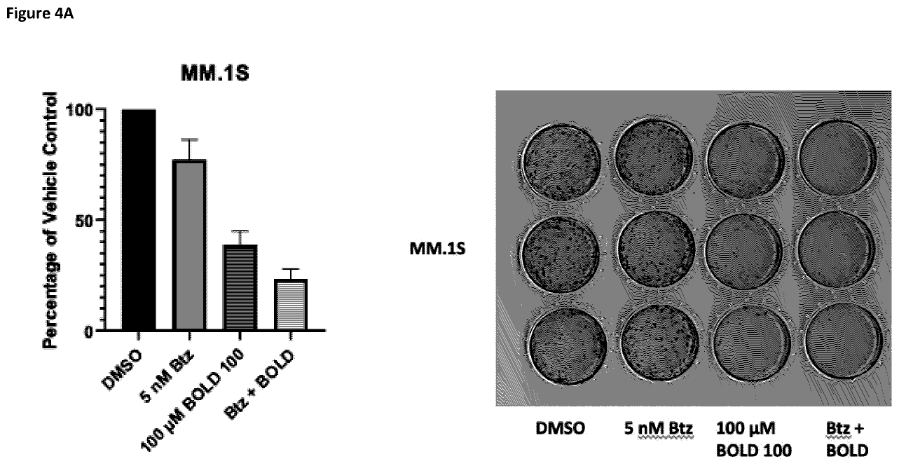

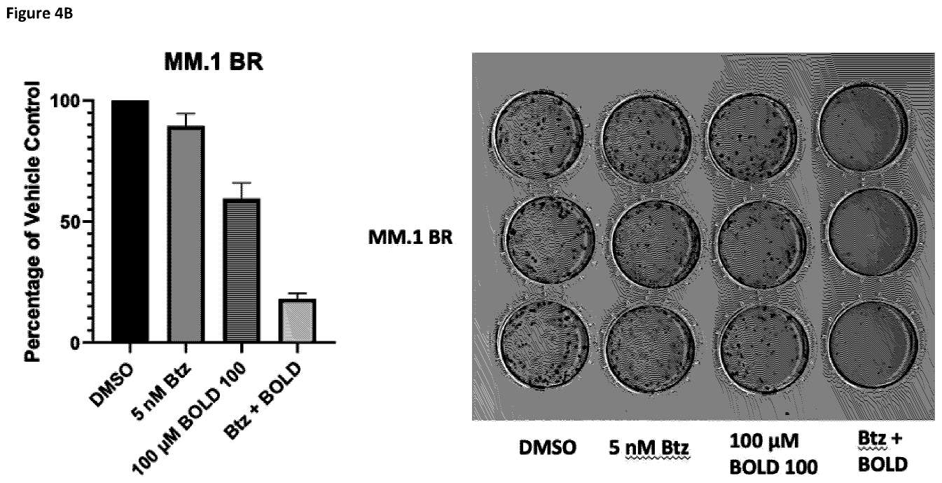

The effect of BOLD-100 in combination with proteasome inhibitors was also demonstrated in a colony forming assay where BOLD-100+PI was tested in both PI sensitive and PI resistant cell lines in a clonogenic assay. The design follows standard methods used in hematopoietic cells. In particular, the cells and drugs were mixed together with semisolid media and plated sparsely to allow individual cells to form colonies. The cells were then allowed to grow into colonies in the semisolid media for a week after which the colonies were stained with crystal violet and images were captured as shown in the FIG. 4A and FIG. 4B. FIG. 4 illustrates that when measured over a longer time period there was a strong negative impact of BOLD-100 in combination with PI on cell viability, and in this assay, we see prevention of regrowth of colonies, demonstrating a strong cytotoxic effect of the drug combination in both sensitive and resistant cell lines.

### Example 5: BOLD-100's Potency Maintained in Co-Culture with Bone Marrow Derived Stromal Cells

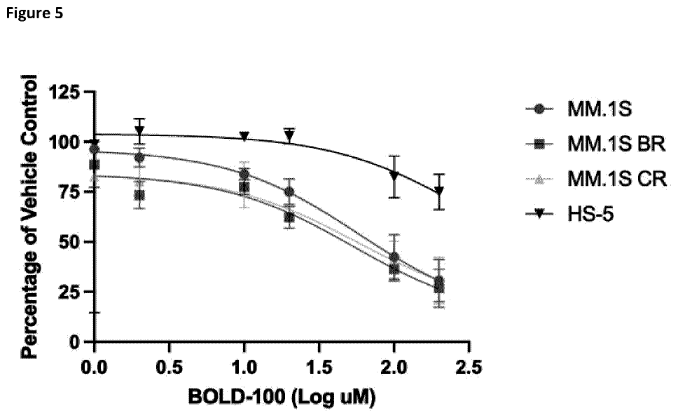

Bone marrow stromal cells are understood to provide a protective environment for cells, insulating bone marrow cells from drug effects. This Example illustrates that BOLD-100 maintains its potency in PI-sensitive and PI-resistant MM cell lines in presence of the bone marrow stromal cells, an environment that partly mimics the in vivo environment. To do this, a co-culture assay was carried out, in which MM cells were grown in co-culture with a bone marrow stromal cell line HS-5. MM cell lines adhere to stromal cells in culture and the stromal cells can provide a protective effect against drug induced apoptosis/growth inhibition. Dose response curves were generated for the PI-sensitive cell line MM.1 S, and the two PI resistant lines MM.1S BR and MM.1S CR in presence of HS-5 by standard CTG assay. As shown in FIG. 5, BOLD-100 was able to negatively impact the viability of all three MM.1S cell lines, but had minimal impact on the bone marrow cell line HS-5.

### REFERENCES

Chou T C. Drug combination studies and their synergy quantification using the Chou-Talalay method. Cancer Res. 2010 Jan. 15;70 (2): 440-6.
Fricker L D. Proteasome Inhibitor Drugs. Annu Rev Pharmacol Toxicol. 2020 Jan. 6; 60:457-476.
Manasanch, E., Orlowski, R. Proteasome inhibitors in cancer therapy. Nat Rev Clin Oncol 14, 417-433 (2017).
Robak, P., Robak, T. Bortezomib for the Treatment of Hematologic Malignancies: 15 Years Later. Drugs R D 19, 73-92 (2019).
Burris, H. A. et al. Safety and activity of IT-139, a ruthenium-based compound, in patients with advanced solid tumours: a first-in-human, open-label, dose-escalation phase I study with expansion cohort. ESMO open. 1, e000154 (2016).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Terms such as “exemplary” or “exemplified” are used herein to mean “serving as an example, instance, or illustration.” Any implementation described herein as “exemplary” or “exemplified” is accordingly not to be construed as necessarily preferred or advantageous over other implementations, all such implementations being independent embodiments. Unless otherwise stated, numeric ranges are inclusive of the numbers defining the range, and numbers are necessarily approximations to the given decimal. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as hereinbefore described and with reference to the examples and drawings.

In some embodiments, the invention excludes steps that involve medical or surgical treatment.

1. A method for treating a disease that is a cancer or a lymphoproliferative disorder in a human patient in need thereof, comprising administering to the patient an effective amount of sodium trans-[tetrachloridobis(1H-indazole)ruthenate(III)] and a combinatorially effective amount of a proteasome inhibitor (PI).

2. A method of ameliorating therapeutic resistance to a proteasome inhibitor (PI) in a disease in a patient in need thereof, comprising administering to the patient an effective amount of sodium trans-[tetrachloridobis(1H-indazole)ruthenate(III)].

3. The method of claim 2, further comprising administering to the patient a combinatorially effective amount of the PI.

4. The method of claim 1 or 3, wherein the effective amount of sodium trans-[tetrachloridobis(1H-indazole)ruthenate(III)] and the combinatorially effective amount of the PI are synergistically effective.

5. The method of any one of claims 1-4, wherein the disease is resistant to treatment with the PI, or the disease is susceptible to developing resistance to the PI.

6. The method of any one of claims 1-5, wherein the PI is bortezomib, carfilzomib, ixazomib citrate, marizomib, oprozomib, delanzomib, ONX 0914 or KZR 616.

7. The method of any one of claims 1-5, wherein the PI is an inhibitor of the constitutive proteasome.

8. The method of any one of claims 1-5, wherein the PI is an inhibitor of the immunoproteasome.

9. The method according to any one of claims 1-8, wherein the disease is a myeloma.

10. The method according to any one of claims 1-8, wherein the disease is a multiple myeloma (MM).

11. The method of claim 9, wherein the MM is relapsing/refractory MM.

12. The method of any one of claims 1-8, wherein the disease is an amyloidosis, a lymphoproliferative disorder, a plasma cell dyscrasia or a lymphoid malignancy.

13. The method of any one of claims 1-8, wherein the disease is a mantle-cell lymphoma, a Waldenstrom macroglobulinemia, or a T-cell lymphoma amyloidosis.

14. The method of claim 1, wherein the cancer is a relapsing/refractory multiple myeloma and the PI is bortezomib or carfilzomib.

15. Use an effective amount of sodium trans-[tetrachloridobis(1H-indazole) ruthenate (III)] in combination with a combinatorially effective amount of a proteasome inhibitor (PI) for treating, or for formulating a medicament to treat, a disease that is a cancer or a lymphoproliferative disorder.

16. Use of amount of sodium trans-[tetrachloridobis(1H-indazole)ruthenate(III)] for ameliorating therapeutic resistance to a proteasome inhibitor (PI) in a disease in a patient in need thereof.

17. The use according to claim 16, wherein the sodium trans-[tetrachloridobis(1H-indazole) ruthenate (III)] is for combined use with the PI.

18. The use according to claim 15 or 17, wherein the effective amount of sodium trans-[tetrachloridobis(1H-indazole)ruthenate(III)] and the combinatorially effective amount of the PI are synergistically effective.

19. The use according to any one of claims 15-18, wherein the disease is resistant to treatment with the PI, or the disease is susceptible to developing resistance to the PI.

20. The use according to any one of claims 15-19, wherein the PI is bortezomib, carfilzomib, ixazomib citrate, marizomib, oprozomib, delanzomib, ONX 0914 or KZR 616.

21. The use according to any one of claims 15-19, wherein the PI is an inhibitor of the constitutive proteasome.

22. The use according to any one of claims 15-19, wherein the PI is an inhibitor of the immunoproteasome.

23. The use according to any one of claims 15-22, wherein the disease is a myeloma.

24. The use according to any one of claims 15-22, wherein the disease is a multiple myeloma (MM).

25. The use according to of claim 24, wherein the MM is relapsing/refractory MM.

26. The use according to any one of claims 15-22, wherein the disease is an amyloidosis, a lymphoproliferative disorder, a plasma cell dyscrasia or a lymphoid malignancy.

27. The use according to any one of claims 15-22, wherein the disease is a mantle-cell lymphoma, a Waldenstrom macroglobulinemia, or a T-cell lymphoma amyloidosis.

28. The use according to claim 15, wherein the cancer is a relapsing/refractory multiple myeloma and the PI is bortezomib or carfilzomib.

29. A method of treating a myeloma that is resistant to a therapeutic, comprising administering to the patient an effective amount of sodium trans-[tetrachloridobis(1H-indazole) ruthenate (III)], wherein the therapeutic is effective to treat myelomas that are not resistant.

30. The method of claim 29, wherein the therapeutic is a proteasome inhibitor (PI), an immunomodulatory imide drug, a cereblon modulator drug, thalidomide or a thalidomide analogue.

31. The method of claim 30, wherein the PI is bortezomib, carfilzomib, ixazomib citrate, marizomib, oprozomib, delanzomib, ONX 0914 or KZR 616.

32. The method of claim 30, wherein the PI is an inhibitor of the constitutive proteasome.

33. The method of claim 30, wherein the PI is an inhibitor of the immunoproteasome.

34. The method of claim 30, wherein the thalidomide analogue is lenalidomide, pomalidomide or iberdomide.

35. The method according to any one of claims 29-34, wherein the myeloma is a multiple myeloma (MM).

36. The method of claim 35, wherein the MM is relapsing/refractory MM.
